(19) United States
(12) Patent Application Publication
(10) Pub. No.: US 2025/0046454 A1
(43) Pub. Date: Feb. 6, 2025

(54) COMPUTER-IMPLEMENTED METHOD FOR PERFORMING A CLINICAL PREDICTION

(71) Applicant: Roche Molecular Systems, Inc., Pleasanton, CA (US)

(72) Inventors: Eldad Klaiman, Starnberg (DE); Amal Lahiani, Munich (DE)

(21) Appl. No.: 18/720,300

(22) PCT Filed: Dec. 15, 2022
(86) PCT No.: PCT/EP2022/086023

§ 371 Date: Jun. 14, 2024

(30) Foreign Application Priority Data
Dec. 17, 2021 (EP) 21215586.5

(51) Int. Cl. G16H 50/20; G16H 30/40; G16H 50/70

(52) U.S. Cl. CPC G16H 50/20; G16H 30/40; G16H 50/70

(57) ABSTRACT

A computer-implemented method for performing a clinical prediction is disclosed. The method comprises the following steps: i) (110) retrieving input data via at least one communication interface (164) of a processing device (166), wherein the input data comprises multiple different modalities of a patient: ii) (114) processing the input data by using the processing device (166), wherein the processing comprises generating embedding modality representations from the input data by using at least one trainable data embedder, wherein the processing comprises combining the embedding modality representations using at least one aggregation network thereby generating the clinical prediction, wherein the aggregation network comprises at least one attention layer and/or at least one transformer layer; and iii) (118) generating an output of the clinical prediction by using the processing device (166).

### TECHNICAL FIELD

The present invention refers to a computer implemented method for performing a clinical prediction, a computer program and a computer-readable storage medium for performing the method according to the present invention. The method and devices, in particular, may be used in the field of clinical research and drug development. Other fields of application of the present invention, however, are feasible.

### BACKGROUND ART

In the field of clinical research and drug development there is abundant patient data available from various sources and in different modalities. Typical data types available are patient clinical data, whole slide microscopic images of biopsies and surgical specimens, gene expression data, proteomics, radiology images such as magnetic resonance imaging (MRI) and computed tomography (CT), and demographics. Personalized health care aims to identify and match the best drugs to the patients they can most benefit from. To do this, algorithms are being developed in order to predict patient survival and response to specific treatments. Other algorithms can be developed to predict or confirm a patient diagnosis or curate and/or complete patient data by predicting missing patient data points.

Until now, these types of predictive algorithms have been typically trained on just one modality of input data to avoid the complexity inherent in the process of combining different types of data modalities. However each modality can include a different information and each of these information can bring additional value to the final prediction and to the performance of the system. Additionally using multiple modalities can be a better approxima-tion of clinicians' behavior when analyzing patient data. Hence combining different patient modalities can improve the quality of the prediction system and can help gain more trust from experts.

Methods for multimodal fusion are known from e.g. Cheerla, Anika, and Olivier Gevaert. “Deep learning with multimodal representation for pancancer prognosis prediction.” Bioin-formatics 35.14 (2019): 1446-i454, Vale-Silva, Luís A., and Karl Rohr. “Long-term cancer survival prediction using multimodal deep learning.” Scientific Reports 11.1 (2021): 1-12, and Sun, Li, et al. “Brain tumor segmentation and survival prediction using multimodal MRI scans with deep learning.” Frontiers in neuroscience 13 (2019): 810.

Despite the achievements of the known methods for multimodal fusion, a suitable mechanism for data multimodal fusion is still challenging, as in Multiple Instance Learning (MIL), the fusion of the multimodal representations (instance representations in MIL) is a crucial step in the algorithm and can greatly impact performance and accuracy.

### Problem to be Solved

It is therefore desirable to provide a method which addresses the above-mentioned technical challenges. Specifically, a method shall be proposed which allows for increased performance and accuracy for data multimodal fusion.

### SUMMARY

This problem is addressed by a computer-implemented method for performing a clinical prediction with the features of the independent claim. Advantageous embodiments which might be realized in an isolated fashion or in any arbitrary combinations are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the invention, a computer implemented method for performing a clinical prediction is disclosed.

The term “computer implemented method” as used herein is a broad term and is to be given its ordinary and customary meaning to a person of ordinary skill in the art and is not to be limited to a special or customized meaning. The term specifically may refer, without limitation, to a method involving at least one computer and/or at least one computer network or a cloud. The computer and/or computer network and/or a cloud may comprise at least one processor which is configured for performing at least one of the method steps of the method according to the present invention. Preferably each of the method steps is performed by the computer and/or computer network and/or a cloud. The method may be performed completely automatically, specifically without user interaction. The term “automatically” as used herein is a broad term and is to be given its ordinary and customary meaning to a person of ordinary skill in the art and is not to be limited to a special or customized meaning. The term specifically may refer, without limitation, to a process which is performed completely by means of at least one computer and/or computer network and/or a cloud and/or machine, in particular without manual action and/or interaction with a user.

The term “clinical prediction” as used herein is a broad term and is to be given its ordinary and customary meaning to a person of ordinary skill in the art and is not to be limited to a special or customized meaning. The term specifically may refer, without limitation, to an estimation of at least one patient endpoint. The patient endpoint may comprise one or more of a measure for efficacy of at least one treatment, a measure for tolerability of at least one treatment, a measure for usefulness of at least one treatment, a measure for harmfulness of at least one treatment, mortality, morbidity, side effects, health-related quality of life and the like. The clinical prediction may comprise at least one expected value. For example, the clinical prediction may comprise one or more of predicting a response to a treatment for a disease, predicting a risk of the patient having the disease, predicting an outcome, derivative a biomarker, and identifying a target for drug development, etc. Disclosed techniques can be used for treatment for different disease types such as different cancer types, and/or to answer other clinical questions, etc. For example, the clinical prediction may comprise predicting whether the patient may be resistant or sensitive to drug treatment, e.g. for cancer. For example, the clinical prediction may comprise predicting a survival rate of the patient for different types of treatments (e.g., immunotherapy, chemotherapy, etc.) for a disease such as cancer. The techniques can also be applied to other disease areas and for other clinical hypotheses. The clinical prediction may be generated and/or provided as a histogram, e.g. showing a development of at least one variable indicative of or relating to the clinical prediction in time.

The term “patient” as used herein is a broad term and is to be given its ordinary and customary meaning to a person of ordinary skill in the art and is not to be limited to a special or customized meaning. The term specifically may refer, without limitation, to a human being or an animal, independent from the fact that the human being or animal, respectively, may be in a healthy condition or may suffer from one or more diseases.

20) The method comprises the following steps which, as an example, may be performed in the given order. It shall be noted, however, that a different order is also possible. Further, it is also possible to perform one or more of the method steps once or repeatedly. Further, it is possible to perform two or more of the method steps simultaneously or in a timely overlapping fashion. The method may comprise further method steps which are not listed.

The method comprises the following steps
i) retrieving input data via at least one communication interface of a processing device, wherein the input data comprises multiple different modalities of a patient;
ii) processing the input data by using the processing device, wherein the processing comprises generating embedding modality representations from the input data by using at least one trainable data embedder, wherein the processing comprises combining the embedding modality representations using at least one aggregation network thereby generating the clinical prediction, wherein the aggregation network comprises at least one attention layer and/or at least one transformer layer; and 35
iii) generating an output of the clinical prediction by using the processing device.

The term “processing device” as generally used herein is a broad term and is to be given its ordinary and customary meaning to a person of ordinary skill in the art and is not to be limited to a special or customized meaning. The term specifically may refer, without limi-tation, to an arbitrary logic circuitry configured for performing basic operations of a computer or system, and/or, generally, to a device which is configured for performing calcula-tions or logic operations. In particular, the processing device may be configured for processing basic instructions that drive the computer or system. As an example, the processing device may comprise at least one arithmetic logic unit (ALU), at least one floating-point unit (FPU), such as a math co-processor or a numeric coprocessor, a plurality of registers, specifically registers configured for supplying operands to the ALU and storing results of operations, and a memory, such as an L1 and L2 cache memory. In particular, the processing device may be a multi-core processor. Specifically, the processing device may be or may comprise a central processing unit (CPU) or Graphics Processing Unit (GPU). Additionally or alternatively, the processing device may be or may comprise a microproces-sor, thus specifically the processing device's elements may be contained in one single integrated circuitry (IC) chip. Additionally or alternatively, the processing device may be or may comprise one or more application-specific integrated circuits (ASICs) and/or one or more field-programmable gate arrays (FPGAs) or the like. The processing device specifically may be configured, such as by software programming, for performing one or more evaluation operations.

The term “communication interface” as used herein is a broad term and is to be given its ordinary and customary meaning to a person of ordinary skill in the art and is not to be limited to a special or customized meaning. The term specifically may refer, without limitation, to an item or element forming a boundary configured for transferring information. In particular, the communication interface may be configured for transferring information from a computational device, e.g. a computer, such as to send or output information, e.g. onto another device. Additionally or alternatively, the communication interface may be configured for transferring information onto a computational device, e.g. onto a computer, such as to receive information. The communication interface may specifically provide means for transferring or exchanging information. In particular, the communication interface may provide a data transfer connection, e.g. Bluetooth, NFC, inductive coupling or the like. As an example, the communication interface may be or may comprise at least one port comprising one or more of a network or internet port, a USB-port and a disk drive.

The communication interface may further comprise at least one display device. The communication interface may be at least one web interface.

The term “retrieving” as used herein is a broad term and is to be given its ordinary and customary meaning to a person of ordinary skill in the art and is not to be limited to a special or customized meaning. The term specifically may refer, without limitation, to the process of a system, specifically a computer system, generating data and/or obtaining data from an arbitrary data source, such as from a data storage, from a network or from a further computer or computer system. The retrieving specifically may take place via at least one computer interface, such as via a port such as a serial or parallel port. The retrieving may comprise several sub-steps, such as the sub-step of obtaining one or more items of primary information and generating secondary information by making use of the primary information, such as by applying one or more algorithms to the primary information, e.g. by using a processor. The retrieving may comprise performing at least one measurement using at least one medical device, e.g. magnetic resonance imaging (MRI), computed tomography (CT) and the like.

The term “input data” as used herein is a broad term and is to be given its ordinary and customary meaning to a person of ordinary skill in the art and is not to be limited to a special or customized meaning. The term specifically may refer, without limitation, to data such as at least one value, parameter, image data, and the like, which can be processed in step ii).

The input data comprises multiple different modalities of a patient. The input data may comprise multimodal clinical data. The term “modality” as used herein is a broad term and is to be given its ordinary and customary meaning to a person of ordinary skill in the art and is not to be limited to a special or customized meaning. The term specifically may refer, without limitation, to a channel of input, e.g. an independent channel of input. The 25 input data may comprise a single datapoint from a single modality or multiple datapoints from the same modality for a single patient. The multiple modalities of a patient may comprise one or more of at least one histology tissue image, at least one whole slide microscopic image of a biopsy and/or a surgical specimen, radiology images such as magnetic resonance imaging (MRI) and computed tomography (CT), genomic data, gene expression data, proteomics, patient clinical data and demographics. The multimodal clinical data may generally refer to clinical data of different types of clinical data, such as molecular data, biopsy image data, etc.

The processing of the input data in step ii) comprises generating embedding modality representations from the input data by using at least one trainable data embedder. The embedding modality representations may be patient level representations, also denoted as patient level embeddings. The term “data embedder”, also denoted as embedder, as used herein is a broad term and is to be given its ordinary and customary meaning to a person of ordinary skill in the art and is not to be limited to a special or customized meaning. The term specifically may refer, without limitation, to at least one network layer configured for converting input data into continuous vector representations. For example, the input data may be an image and the data embedder is designed for converting the image into a lower-dimensional representation of the image. An output of the trainable data embedder may be a generic embedding representation per modality or multiple instance embeddings for each modality.

The term “trainable data embedder” as used herein is a broad term and is to be given its ordinary and customary meaning to a person of ordinary skill in the art and is not to be limited to a special or customized meaning. The term specifically may refer, without limitation, to the fact that the embedder can be further trained and/or updated based on additional training data. Specifically, the embedder is trained on a training dataset. The embedder may be trained using machine learning. At least one embedder may be used for each modality. The respective embedder for a modality may be trained on historical data from the modality. For example, the respective embedder may be trained on historical data from histology tissue imaging, whole slide microscopic imaging, or radiology imaging, or historical genomic data, historical gene expression data, historical proteomics, historical patient clinical data or historical demographics. The embedder may be updated by using new-ly received input data.

The training data may comprise data from a number of patients, each with different data modalities and a known ground truth outcome. For example, the training data may comprise at least one histology whole slide image (e.g. H&E). The slide may have expert anno-tations and/or tissue detection masks. The whole slide image is a high resolution image, and tile images can be extracted from the whole slide and/or from specific expert annota-tions and/or from tissue masks to create the image modality datapoint. The patient can additionally have genomic or proteomic datapoints. These can be vectors of raw or normal-ized floating point values. The training data may comprise patient metadata such as age, gender, etc., and clinical data, e.g. diagnosis, her2 positivity status . . . , etc., The training data may comprise one or multiple patient embeddings generated with a different analysis system.

The processing comprises combining the embedding modality representations using at least one aggregation network thereby generating the clinical prediction. The embedding modality representations may be introduced as input into the attention layer and/or the transformer layer. The term “combining” as used herein is a broad term and is to be given its ordinary and customary meaning to a person of ordinary skill in the art and is not to be limited to a special or customized meaning. The term specifically may refer, without limitation, to data fusion and/or data aggregation. The term “aggregation network” as used herein is a broad term and is to be given its ordinary and customary meaning to a person of ordinary skill in the art and is not to be limited to a special or customized meaning. The term specifically may refer, without limitation, to a deep neural network architecture designed for combining embedding modality representations from a multitude of modalities thereby generating the clinical prediction. The combining may comprise one or more of considering a sum of the transformed datapoints, considering a maximum of the transformed datapoints, using attention MIL models, and using vision transformers.

The aggregation network comprises at least one attention layer and/or at least one transformer layer. The attention layer and/or the transformer layer may run a self-attention mechanism. The self-attention mechanism may allow creating an optimal combination strategy for the multimodal data.

The term “attention layer” as used herein is a broad term and is to be given its ordinary and customary meaning to a person of ordinary skill in the art and is not to be limited to a special or customized meaning. The term specifically may refer, without limitation, to a layer of a neural network designed for enhancing at least one important part of the input data and fading out the other parts. The importance of input data may depend on the modality. The importance of input data may be learned through training data. The attention layer may use dot-product attention and/or multi-head attention.

The term “transformer layer” as used herein is a broad term and is to be given its ordinary and customary meaning to a person of ordinary skill in the art and is not to be limited to a special or customized meaning. The term specifically may refer, without limitation, to a layer of a deep learning model that adopts the mechanism of self-attention, differentially weighting the significance of each part of the input data. For example, the transformer layer may be based on a vision transformer model. A vision transformer model is an image classification model based on the transformer encoder architecture and using embeddings of image patches as inputs. In the vision transformer model, an image may be split into patches, then the patches are flattened, projected into lower dimensional embeddings, add-ed to positional embeddings and fed to a transformer encoder network. The output of the transformer encoder may be used as an input to a Multi-Layer Perceptron (MLP) head to generate the final prediction. The MLP head may comprise a set of linear transformation layers. The transformer encoder may comprise n encoders. Each encoder may comprise a multi-headed attention layer, normalization layers and an MLP layer. The term “Multi-Layer Perceptron neural network” or “MLP neural network” as used herein is a broad term and is to be given its ordinary and customary meaning to a person of ordinary skill in the art and is not to be limited to a special or customized meaning. The term specifically may refer, without limitation, to a class of feedforward artificial neural networks. Residual skip connexions may be additionally used between the encoder's sublayers to enable the interaction between different level representations and prevent the vanishing gradient problem. Multi-headed attention may be based on running the self-attention mechanism multiple times. With respect to further design of the Multi-headed attention reference can be made to Vaswani, A., Shazeer, N., Parmar, N., Uszkoreit, J., Jones, L., Gomez, A. N., . . . & Po-losukhin, I. (2017), “Attention is all you need in Advances in neural information processing systems”, pp. 5998-6008. Self attention is a mechanism allowing to learn the relationships between the different inputs and to take these relationships into consideration during model training. Using a vision transformer as a secondary network to aggregate different modality embeddings is a new way of using this category of models. The present invention proposes to find relevant relationships between the different modalities and use it as an additional information during training.

The present invention proposes to use a deep neural network architecture dedicated to predicting patient endpoints from a multitude of modalities. Specifically, the present invention proposes to fuse the embedding modality representations, also denoted as multimodal data representations, by using an attention based pooling method for multimodal representation fusion and/or a transformer based pooling method for multimodal representation fusion. The multimodal data representations of a patient may be introduced as input into the attention layer and/or transformer layer which may learn through backpropagation the optimal combination and/or attention strategy (parameters) for this dataset. The proposed network architecture may utilize an attention MIL module and/or a transformer module architecture. This may allow creating an optimal combination strategy for the multimodal data via a self-attention mechanism, e.g. available in the transformer module architecture.

MIL networks are a class of weakly supervised learning models where training instances are grouped into bags and labels are assigned to the bags instead of single instances. Attention based MIL networks may allow to aggregate different instance outputs according to an attention mechanism and hence to assess the contribution of each instance in the bag to final bag output. Both prediction and attention networks may be learned simultaneously.

The method may comprise the attention layer and/or the transformer layer learning, for example training and/or optimizing, through backpropagation an optimal combination and/or attention strategy, e.g. for determining the parameters for a dataset.

The input data may comprise at least one datapoint from each of the multiple different modalities. For example, the at least one datapoint may comprise at least one image tile from a single biopsy, at least one gene seriousness, at least one tile from a radiology image, etc., The input data may comprise multiple datapoints from the same modality for a single patient e.g. multiple image tiles from a single biopsy, multiple gene seriousness, multiple tiles from a radiology image, etc., For example, the input data may comprise a single datapoint from each of the multiple different modalities. For example, the input data may comprise multiple data points from each of the multiple different modalities. For example, the input data may comprise a single datapoint from one or more of the modalities and multiple data points from at least one of the other modalities.

The method may comprise generating an embedding modality representation from each datapoint. For example, in case of a single datapoint from each of the different modalities, the method may comprise generating an embedding modality representation from each of the single datapoints. The method may further comprise generating from the embedding modality representations of the different modalities the clinical prediction using the aggregation network.

The method may comprise generating an embedding modality representation from each datapoint, combining the generated embedding modality representations for each of the modalities separately, and generating from the combined embedding modality representations a clinical prediction using the aggregation network.

For example, the input data may comprise multiple datapoints from the same modality for a single patient. The method may comprise generating an embedding modality representation from each datapoint, combining the generated embedding modality representations for each of the different modalities separately, and generating from the combined embedding modality representations a clinical prediction using the aggregation network. Additionally or alternatively, the method may comprise combining the multiple datapoints, generating a global embedding modality representation from the combined datapoints for each of the different modalities, and generating from the global embedding modality representations a clinical prediction using the aggregation network.

The method may comprise combining the multiple datapoints, generating a global embedding modality representation from the combined datapoints for each of the modalities, and generating from the global embedding modality representations a clinical prediction using the aggregation network. For example, in case of multiple datapoints from the same modality for a single patient, the method may comprise creating an embedding from each datapoint, combining the embeddings for each of the modalities separately, and then using a network layer to convince the patient level multimodal datapoints into a patient level prediction. Additionally or alternatively, in case of multiple datapoints from the same modality for a single patient, the multiple datapoints from multimodal data may be directly combined together and a network layer may be used to convince them into the patient level prediction without first combining them into one patient level modality datapoint.

In one embodiment, the different modalities are converted to patient level embeddings by a primary attention MIL network layer and then input into a secondary attention mil network that combines the multimodal patient level data into a patient level prediction. Using back propagation, the network may be trained and optimized.

In one embodiment, the different modalities are converted to patient level embeddings by a primary attention MIL network layer and then input into a secondary vision transformer network that combines the multimodal patient level data into a patient level prediction. Using back propagation, the network may be trained and optimized.

In one embodiment, the different modalities are converted to patient level embeddings by a primary vision transformer network layer and then input into a secondary vision transformer network that combines the multimodal patient level data into a patient level prediction. Using back propagation, the network may be trained and optimized.

In one embodiment, the different modalities are converted to patient level embeddings by a primary vision transformer network layer and then input into a secondary attention MIL network that combines the multimodal patient level data into a patient level prediction. Using back propagation, the network may be trained and optimized.

In one embodiment, the different modalities are directly input into an embedder network and the resulting embeddings are input into a primary attention MIL network layer that combines the multimodal raw data into a patient level prediction. Using back propagation, the network is trained and optimized.

In one embodiment, the different modalities are directly input into an embedder network and the resulting embeddings are input into a primary vision transformer network layer that combines the multimodal raw data into a patient level prediction. Using back propagation, the network is trained and optimized.

In one embodiment, depending on the data type, each modality can be converted to patient level embeddings by a primary attention mil network layer or directly input into an embedder network. All resulting embeddings are input into a secondary attention mil network that combines the multimodal data into a patient level prediction. Using back propagation, the network may be trained and optimized.

In one embodiment, depending on the data type, each modality can be converted to patient level embeddings by a primary attention mil network layer or directly input into an embedder network. All resulting embeddings are input into a secondary vision transformer network that combines the multimodal data into a patient level prediction. Using back propagation, the network may be trained and optimized.

The method may further comprise at least one preprocessing step. The preprocessing step may comprise transforming raw data into a new format. This step may depend on the modality type. For example, it is possible to project the histology tile images into a different space by using a pre-trained embedder. This can help capture relevant information in the raw data and potentially speed the training process.

The output of the clinical prediction may comprise one or more of information about drugs for the patient, information about patient survival, information about response to at least one specific treatment, information confirming a patient diagnosis, information curating and/or completing patient data by predicting missing patient data points. The method comprises at least one output step comprising providing the clinical prediction via at least one output interface. The term “output interface”, as used herein, relates to any arbitrary unit configured for a transfer of information from the processing device to another entity, wherein another entity may be a further data processing device and/or a user. Thus, the output interface may comprise a user interface, such as an appropriately configured display, or may be a printer.

Further disclosed and proposed herein is a computer program including computer-executable instructions for performing the method according to the present invention in one or more of the embodiments enclosed herein when the instructions are executed on a computer or computer network. Specifically, the computer program may be stored on a computer-readable data carrier and/or on a computer-readable storage medium.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps i) to iii) as indicated above may be performed by using a computer or a computer network, preferably by using a computer program.

Further disclosed and proposed herein is a computer program product having program code means, in order to perform the method according to the present invention in one or more of the embodiments enclosed herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method according to one or more of the embodiments disclosed herein.

Further disclosed and proposed herein is a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the method according to one or more of the embodiments disclosed herein.

Referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

In a further aspect of the present invention, a clinical prediction device is disclosed. The clinical prediction device comprises at least one processing device having at least one communication interface configured for retrieving input data. The input data comprises multiple different modalities of a patient. The processing device is configured for processing the input data. The processing comprises generating embedding modality representations from the input data by using at least one trainable data embedder. The processing comprises combining the embedding modality representations using at least one aggregation network thereby generating the clinical prediction. The aggregation network comprises at least one attention layer and/or at least one transformer layer. The processing device is configured for generating an output of the clinical prediction.

The clinical prediction device may be configured for performing the method for performing a clinical prediction according to the present invention. Thus, with respect to definitions and embodiments of the clinical prediction device reference is made to definitions and embodiments described with respect to the method.

Summarizing and without excluding further possible embodiments, the following embodiments may be envisaged:
Embodiment 1. A computer-implemented method for performing a clinical prediction, comprising
   i) retrieving input data via at least one communication interface of a processing device, wherein the input data comprises multiple different modalities of a patient;
   ii) processing the input data by using the processing device, wherein the processing comprises generating embedding modality representations from the input data by using at least one trainable data embedder, wherein the processing comprises combining the embedding modality representations using at least one aggregation network thereby generating the clinical prediction, wherein the aggregation network comprises at least one attention layer and/or at least one transformer layer; and
   iii) generating an output of the clinical prediction by using the processing device.
Embodiment 2. The method according to the preceding embodiment, wherein the output of the clinical prediction comprises one or more of information about drugs for the patient, information about patient survival, information about response to at least one specific treatment, information confirming a patient diagnosis, information curating and/or completing patient data by predicting missing patient data points.
Embodiment 3. The method according to any one of the preceding embodiments, wherein the method comprises at least one output step comprising providing the clinical prediction via at least one output interface.
Embodiment 4. The method according to any one of the preceding embodiments, wherein an output of the trainable data embedder is a generic patient level embedding representation per modality or multiple instance embeddings for each modality.
Embodiment 5. The method according to any one of the preceding embodiments, wherein the embedding modality representations are introduced as input into the attention layer and/or the transformer layer.
Embodiment 6. The method according to any one of the preceding embodiments, wherein the multiple modalities of a patient comprise one or more of at least one histology tissue image, at least one whole slide microscopic image of a biopsy and/or a surgical specimen, radiology images such as magnetic resonance imaging (MRI) and computed tomography (CT), genomic data, gene expression data, proteomics, patient clinical data and demographics.
Embodiment 7. The method according to any one of the preceding embodiments, wherein the method comprises the attention layer and/or the transformer layer learning through backpropagation an optimal combination and/or attention strategy.
Embodiment 8. The method according to any one of the preceding embodiments, wherein the input data comprises at least one datapoint from each of the different modalities, wherein the method comprises generating an embedding modality representation from each of the datapoints and generating from the embedding modality representations of the different modalities the clinical prediction using the aggregation network.
Embodiment 9. The method according to any one of the preceding embodiments, wherein the input data comprises multiple datapoints from the same modality for a single patient for at least one of the different modalities.
Embodiment 10. The method according to the preceding embodiment, wherein the method comprises generating an embedding modality representation from each datapoint, combining the generated embedding modality representations for each of the different modalities separately, and generating from the combined embedding modality representations the clinical prediction using the aggregation network.
Embodiment 11. The method according to the pre-preceding embodiment, wherein the method comprises combining the multiple datapoints and generating a global embedding modality representation from the combined datapoints for each of the different modalities, and generating from the global embedding modality representations the clinical prediction using the aggregation network.
Embodiment 12. The method according to any one of the preceding embodiments, wherein the different modalities are converted to embedding modality representations by a primary attention MIL network layer and are then input into a secondary attention MIL network that combines the embedding modality representations into a clinical prediction.
Embodiment 13. The method according to any one of the preceding embodiments, wherein the different modalities are converted to embedding modality representations by a primary attention MIL network layer and are then input into a secondary vision transformer network that combines the embedding modality representations into a clinical prediction.
Embodiment 14. The method according to any one of the preceding embodiments, wherein the different modalities are converted to embedding modality representations by a primary vision transformer network layer and then input into a secondary vision transformer network that combines the embedding modality representations into a clinical prediction.
Embodiment 15. The method according to any one of the preceding embodiments, wherein the different modalities are converted to embedding modality representations by a primary vision transformer network layer and are then input into a secondary attention MIL network that combines the embedding modality representations into a clinical prediction.
Embodiment 16. The method according to any one of the preceding embodiments, wherein the different modalities are input into an embedder network and the resulting embedding modality representations are input into a primary attention MIL network layer that combines the embedding modality representations into a clinical prediction.
Embodiment 17. The method according to any one of the preceding embodiments, wherein the different modalities are input into an embedder network and the resulting embedding modality representations are input into a primary vision transformer network layer that combines the multimodal raw data into a clinical prediction.
Embodiment 18 The method according to any one of the preceding embodiments, wherein, depending on the data type, each modality is converted to embedding modality representations by a primary attention MIL network layer or input into an embedder network, wherein the resulting embedding modality representations are input into a secondary attention MIL network that combines the embedding modality representations into a clinical prediction.
Embodiment 19. The method according to any one of the preceding embodiments, wherein, depending on the data type, each modality is converted to embedding modality representations by a primary attention MIL network layer or input into an embedder network, wherein the resulting embedding modality representations are input into a secondary vision transformer network that combines the embedding modality representations into a clinical prediction.
Embodiment 20. The method according to any one of the preceding embodiments, wherein the method comprises at least one preprocessing step, wherein the preprocessing step comprises transforming raw data into a new format.
Embodiment 21. A computer program comprising instructions which, when the program is executed by a processing device, cause the processing device to carry out steps i) to iii) of the method according to any one of the preceding embodiments.
Embodiment 22. A computer-readable storage medium comprising instructions which, when executed by a processing device, cause the processing device to carry out steps i) to iii) of the method according to any one of the preceding embodiments referring to a method.
Embodiment 23. A clinical prediction device comprising at least one processing device having at least one communication interface configured for retrieving input data, wherein the input data comprises multiple different modalities of a patient, wherein the processing device is configured for processing the input data, wherein the processing comprises generating embedding modality representations from the input data by using at least one trainable data embedder, wherein the processing comprises combining the embedding modality representations using at least one aggregation network thereby generating the clinical prediction, wherein the aggregation network comprises at least one attention layer and/or at least one transformer layer, wherein the processing device is configured for generating an output of the clinical prediction.
Embodiment 24. The clinical prediction device according to the preceding embodiment, wherein the clinical prediction device is configured for performing the method for performing a clinical prediction according to any one of the preceding embodiments referring to a method.

### SHORT DESCRIPTION OF THE FIGURES

Further optional features and embodiments will be disclosed in more detail in the subse-quent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 shows an embodiment of a workflow of the method according to the present invention;

FIG. 2 shows a further example workflow with three selected modalities for each patient;

FIG. 3 shows an example of mixed global multimodal transformer based fusion setup;

FIG. 4 shows an architecture of a vision encoder; and

FIGS. 5A to 5D show further examples of the method according to the present invention.

### DETAILED DESCRIPTION OF THE EMBODIMENTS

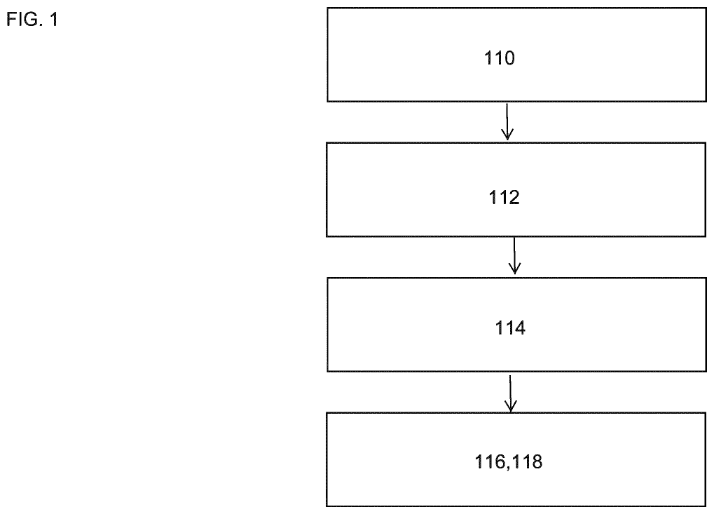

FIG. 1 shows the general high level workflow of the computer-implemented method for performing a clinical prediction according to the present invention. In this embodiment, step i) 110 comprises receiving multiple modalities for each patient. The modalities can be of different types and can hold different information e.g. histology tissue images (e.g. H&E and/or IHC and/or fluorescence stained slide images), gene sequence data (e.g. RNA-Seq, mRNA), clinical data (e.g. tumor type, tissue type). In FIG. 1 a preprocessing step 112 is shown comprising transforming the original raw data into a new format. This step 112 is optional and depends on the modality type. For example, it is possible to project the histology tile images into a different space by using a pre-trained embedder. This can help capture relevant information in the raw data and potentially speed the training process. Step ii) 114 may comprise inputting each of the modalities into a trainable data embedder in order to obtain meaningful embedding modality representations. In this step 114, the output can be either a generic patient level embedding representation per modality or multiple instance embeddings for each modality depending on the nature of the data and the chosen embodiment. All these output embeddings are then combined in step 116 using an aggregation network. Many aggregation options exist e.g. mean or max operators, attention networks, transformers. Further, step iii) 118 comprises generating an output of the clinical prediction.

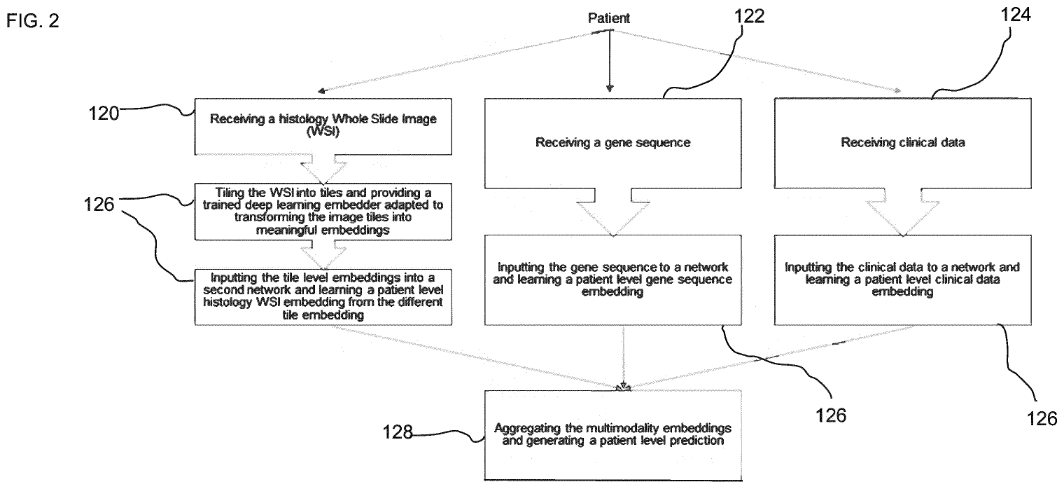

FIG. 2 shows an example workflow with three selected modalities for each patient: a histology whole slide image (WSI) 120, a gene sequence 122 and clinical data 124. The method may comprise generating an embedding modality representation from each datapoint, combining the generated embedding modality representations for each of the modalities separately, and generating from the combined embedding modality representations a clinical prediction using the aggregation network. In this example, each modality goes through a set of steps 126 for generating an embedding modality representation. For the WSI 120, as high resolution WSIs are typically very large and cannot be fitted into memory, the method may comprise tiling the slide into non overlapping patches. Then each patch is projected into a different space using a pre-trained embedder (e.g. Resnet). These tile level embeddings are inputted to a trainable aggregation network (e.g. attention MIL) to generate a patient level embedding of the WSI. The gene sequence 122 and the clinical data 124 are each passed to a trainable embedder to generate a representative patient level embedding for each of the modalities. All patient level embeddings are then passed to a second aggregation model 128 to generate a global patient level embedding and a final patient prediction.

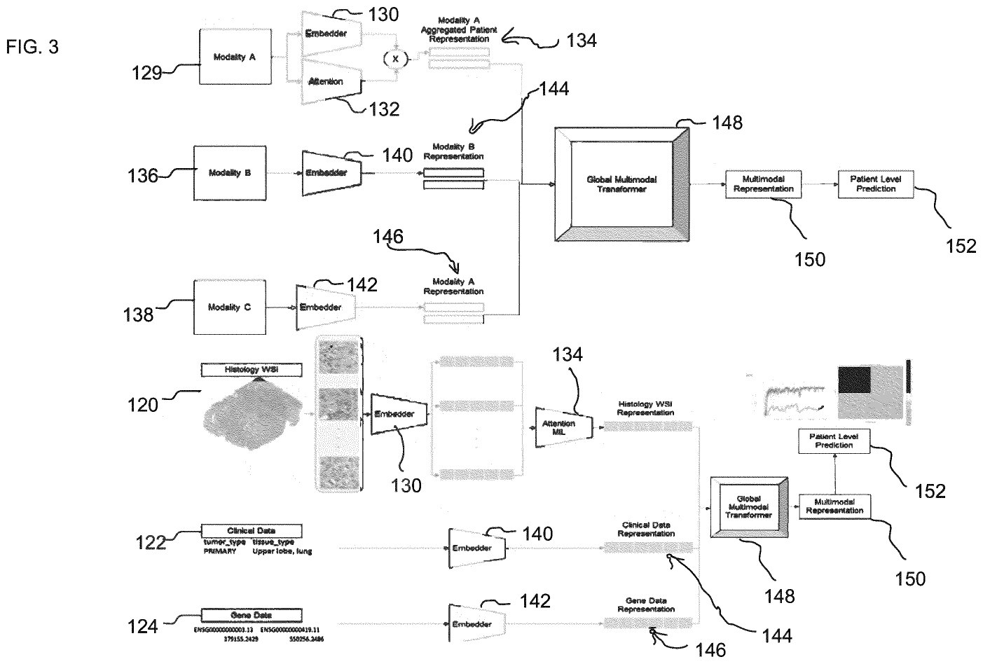

FIG. 3 shows an example of mixed global multimodal transformer based fusion setup. In the upper part of FIG. 3 a general example of the used components is shown and in the lower part an application using three selected modalities for each patient: a histology whole slide image (WSI) 120, a gene sequence 122 and clinical data 124. Modality A 129 (WSI 120 in the lower part of FIG. 3) may go through an embedding projection, e.g. embedder 130, and a trainable attention network 132 to generate a patient level representation 134. Modalities B 136 (clinical data 124 in the lower part of FIG. 3) and C 138 (gene sequence 122 in the lower part of FIG. 3) may go through trainable embedders 140, 142 to generate patient level representations 144, 146. The patient level representations 134, 144, 146 are then passed to a global multimodal vision transformer 148 to generate a multimodal patient representation 150 and a patient level prediction 152.

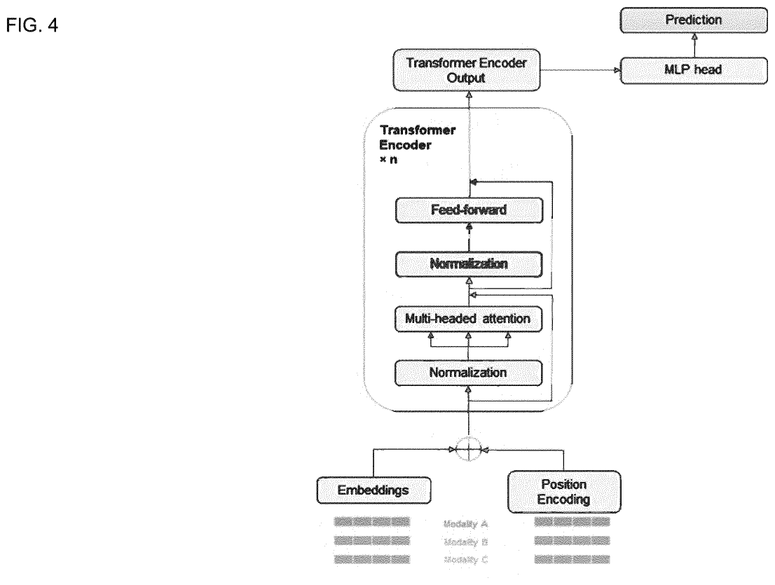

FIG. 4 shows the architecture of a transformer encoder as described in Dosovitskiy, A., Beyer, L., Kolesnikov, A., Weissenborn, D., Zhai, X., Unterthiner, T., . . . & Houlsby, N. (2020). An image is worth 16×16 words: Transformers for image recognition at scale. arXiv preprint arXiv: 2010.11929.

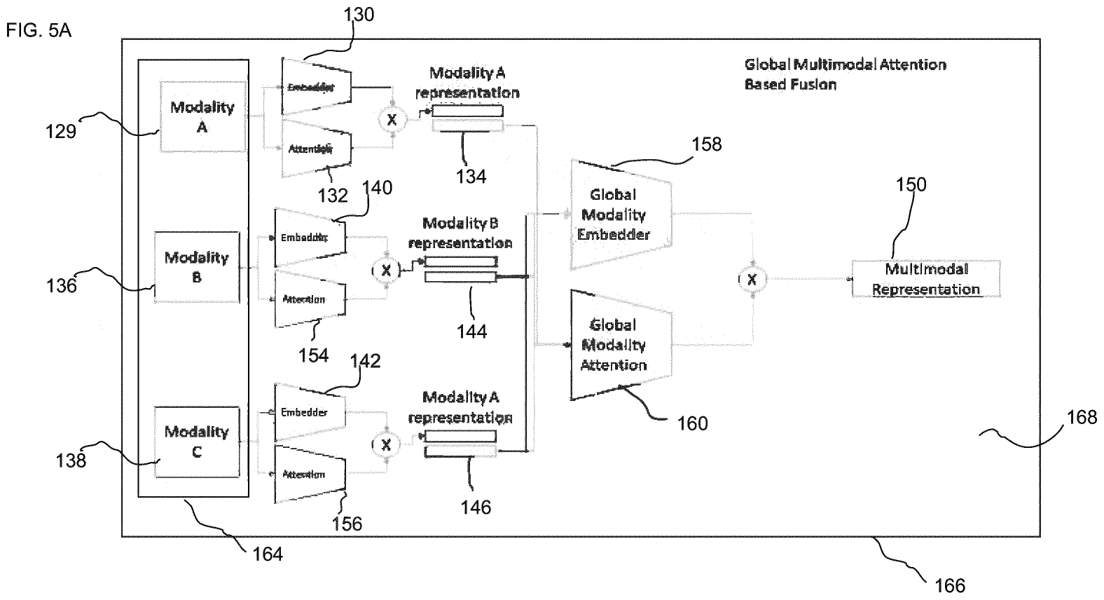

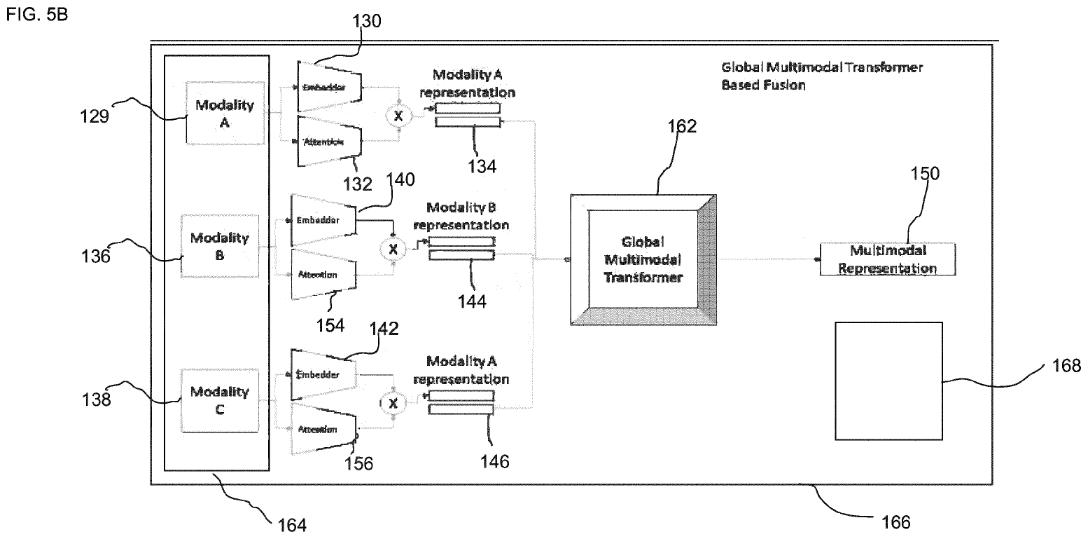

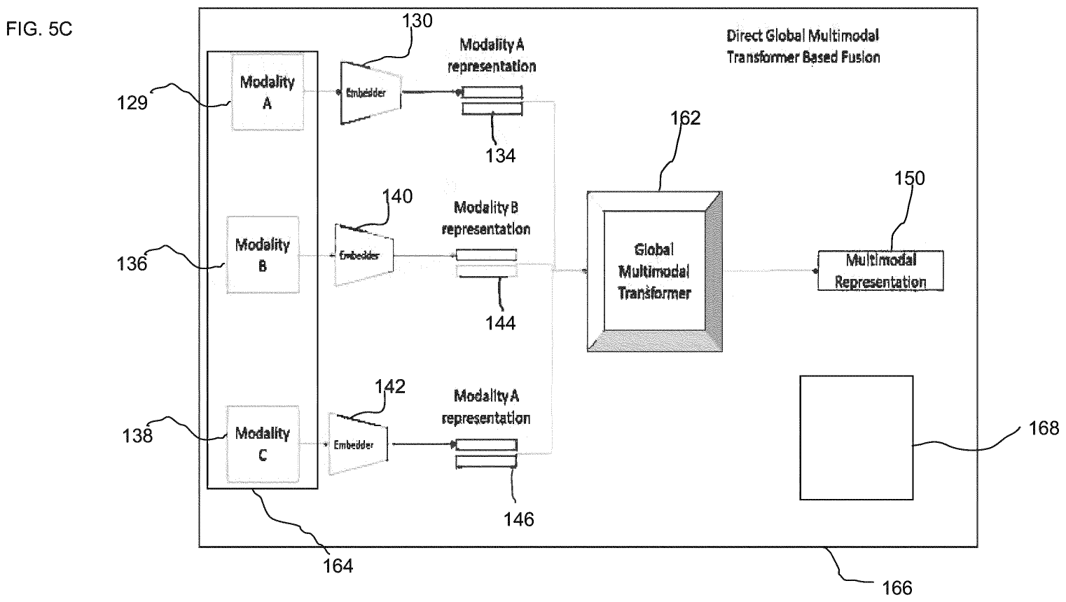

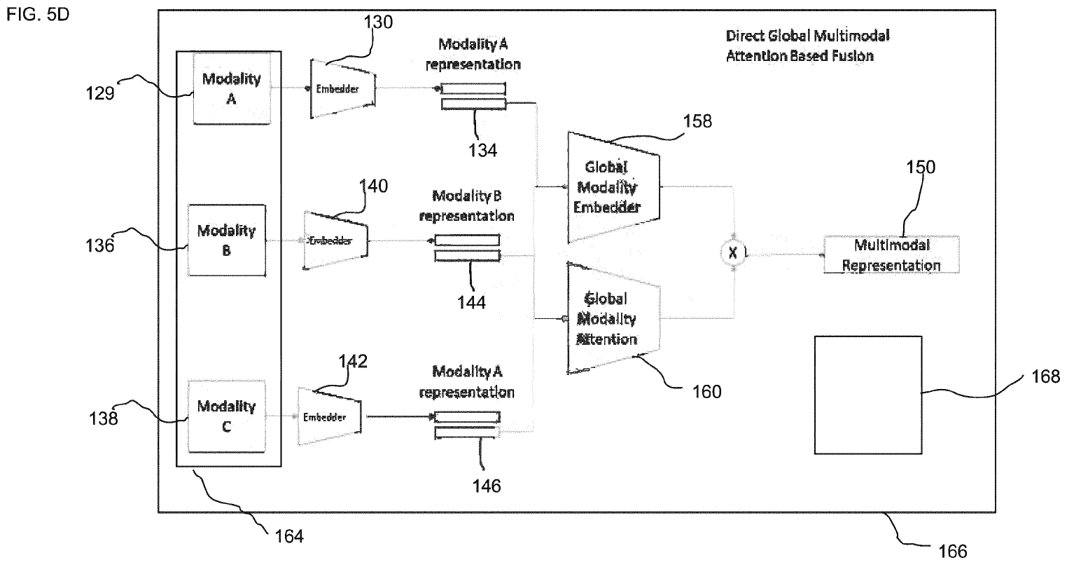

FIGS. 5A to 5D show further examples of the method according to the present invention for three modalities.

FIG. 5A shows received modality A 129, modality B 136 and modality C 138. Modality A 129, modality B 136 and modality C 138 may each go through an embedder 130, 140, 142 and a trainable attention network 132, 154, 156 to generate a modality representation 134, 144, 146 for each of the modalities. The modality representation 134, 144, 146 are then passed to a global multimodality embedder 158 and a global modality attention network 160 to generate a multimodal patient representation 150 and a patient level prediction.

FIG. 5B shows received modality A 129, modality B 136 and modality C 138. Modality A 129, modality B 136 and modality C 138 may each go through an embedder 130, 140, 142 and a trainable attention network 132, 154, 156 to generate a modality representation 134, 144, 146 for each of the modalities. The modality representation 134, 144, 146 are then passed to a global multimodal transformer 162 to generate a multimodal patient representation 150 and a patient level prediction.

FIG. 5C shows received modality A 129, modality B 136 and modality C 138. Modality A 129, modality B 136 and modality C 138 may each go through an embedder 130, 140, 142 to generate a modality representation 134, 144, 146 for each of the modalities. The modality representation 134, 144, 146 are then passed to a global multimodal transformer 162 to generate a multimodal patient representation 150 and a patient level prediction.

FIG. 5D shows received modality A 129, modality B 136 and modality C 138. Modality A 129, modality B 136 and modality C 138 may each go through an embedder 130, 140, 142 to generate a modality representation 134, 144, 146 for each of the modalities. The modality representation 134, 144, 146 are then passed to a global multimodality embedder 158 and a global modality attention network 160 to generate a multimodal patient representation 150 and a patient level prediction.

As shown highly schematically in FIGS. 5A to 5D, the input data is received via at least one communication interface 164 of a processing device 166. The processing device 166 is configured for processing the input data. The clinical prediction may be provided via at least one output interface 168, e.g. of the processing device 166 or a further device such as a display and/or printer. Further shown in FIGS. 5A to 5D is an embodiment of a clinical prediction device 170 comprising the communication interface 164 and the processing device 166. The clinical prediction device 170 may further comprise the output interface 168.

### LIST OF REFERENCE NUMBERS

110 step i)
112 preprocessing step
114 step ii)
116 combining
118 step iii)
120 whole slide image
122 gene sequence
124 clinical data
126 steps for generating an embedding modality representation
generating a global patient level embedding and prediction 126
128 aggregation model
129 modality A
130 embedder
132 trainable attention network
134 modality (patient level) representation
136 modality B
138 modality C
140 embedder
142 embedder
144 modality (patient level) representations
146 modality (patient level) representations
148 global multimodal vision transformer
150 multimodal patient representation
152 patient level prediction
154 attention network
156 attention network
158 global multimodality embedder
160 global modality attention network
162 global multimodal transformer
164 communication interface
166 processing device
168 output interface
170 clinical prediction device

1. Computer-implemented method for performing a clinical prediction, comprising
i) (110) retrieving input data via at least one communication interface (164) of a processing device (166), wherein the input data comprises multiple different modalities of a patient;
ii) (114) processing the input data by using the processing device (166), wherein the processing comprises generating embedding modality representations from the input data by using at least one trainable data embedder, wherein the processing comprises combining the embedding modality representations using at least one aggregation network thereby generating the clinical prediction, wherein the aggregation network comprises at least one attention layer and/or at least one transformer layer; and
iii) (118) generating an output of the clinical prediction by using the processing device (166).

2. The method according to the preceding claim, wherein the output of the clinical prediction comprises one or more of information about drugs for the patient, information about patient survival, information about response to at least one specific treatment, information confirming a patient diagnosis, information curating and/or completing patient data by predicting missing patient data points.

3. The method according to any one of the preceding claims, wherein the method comprises at least one output step comprising providing the clinical prediction via at least one output interface (168), wherein an output of the trainable data embedder is a generic patient level embedding representation per modality or multiple instance embeddings for each modality.

4. The method according to any one of the preceding claims, wherein the multiple modalities of a patient comprise one or more of at least one histology tissue image, at least one whole side microscopic image of a biopsy and/or a surgical specimen, radiology images such as magnetic resonance imaging (MRI) and computed tomography (CT), genomic data, gene expression data, proteomics, patient clinical data and demographics.

5. The method according to any one of the preceding claims, wherein the method comprises the attention layer and/or the transformer layer learning through backpropagation an optimal combination and/or attention strategy.

6. The method according to any one of the preceding claims, wherein the input data comprises at least one datapoint from each of the different modalities, wherein the method comprises generating an embedding modality representation from each of the datapoints and generating from the embedding modality representations of the different modalities the clinical prediction using the aggregation network, and/or
wherein the input data comprises multiple datapoints from the same modality for a single patient for at least one of the different modalities, wherein the method comprises generating an embedding modality representation from each datapoint, combining the generated embedding modality representations for each of the different modalities separately, and generating from the combined embedding modality representations the clinical prediction using the aggregation network, and/or, wherein the method comprises combining the multiple datapoints and generating a global embedding modality representation from the combined datapoints for each of the different modalities, and generating from the global embedding modality representations the clinical prediction using the aggregation network.

7. The method according to any one of the preceding claims, wherein the different modalities are converted to embedding modality representations by a primary attention multiple instance learning (MIL) network layer and are then input into a secondary attention MIL network that combines the embedding modality representations into a clinical prediction.

8. The method according to any one of the preceding claims, wherein the different modalities are converted to embedding modality representations by a primary attention MIL network layer and are then input into a secondary vision transformer network that combines the embedding modality representations into a clinical prediction.

9. The method according to any one of the preceding claims, wherein the different modalities are converted to embedding modality representations by a primary vision transformer network layer and then input into a secondary vision transformer network that combines the embedding modality representations into a clinical prediction.

10. The method according to any one of the preceding claims, wherein the different modalities are converted to embedding modality representations by a primary vision transformer network layer and are then input into a secondary attention MIL network that combines the embedding modality representations into a clinical prediction.

11. The method according to any one of the preceding claims, wherein the different modalities are input into an embedder network and the resulting embedding modality representations are input into a primary attention MIL network layer that combines the embedding modality representations into a clinical prediction.

12. The method according to any one of the preceding claims, wherein the different modalities are input into an embedder network and the resulting embedding modality representations are input into a primary vision transformer network layer that combines the multimodal raw data into a clinical prediction.

13. The method according to any one of the preceding claims, wherein, depending on the data type, each modality is converted to embedding modality representations by a primary attention MIL network layer or input into an embedder network, wherein the resulting embedding modality representations are input into a secondary attention MIL network that combines the embedding modality representations into a clinical prediction.

14. The method according to any one of the preceding claims, wherein, depending on the data type, each modality is converted to embedding modality representations by a primary attention MIL network layer or input into an embedder network, wherein the resulting embedding modality representations are input into a secondary vision transformer network that combines the embedding modality representations into a clinical prediction.

15. A clinical prediction device (170) comprising at least one processing device (166) having at least one communication interface (164) configured for retrieving input data, wherein the input data comprises multiple different modalities of a patient, wherein the processing device (166) is configured for processing the input data, wherein the processing comprises generating embedding modality representations from the input data by using at least one trainable data embedder, wherein the processing comprises combining the embedding modality representations using at least one aggregation network thereby generating the clinical prediction, wherein the aggregation network comprises at least one attention layer and/or at least one transformer layer, wherein the processing device (166) is configured for generating an output of the clinical prediction.
